(19) United States
(12) Patent Application Publication
(10) Pub. No.: US 2021/0316166 A1
(43) Pub. Date: Oct. 14, 2021

(54) APPARATUS FOR IDENTIFYING LEAK OF FACE MASK AND METHOD THEREFOR

(71) Applicant: National Cheng Kung University, Tainan City (TW)

(72) Inventor: Ming-Yeng LIN, Kaohsiung City (TW)

(21) Appl. No.: 17/227,415

(22) Filed: Apr. 12, 2021

Related U.S. Application Data
(60) Provisional application No. 63/008,817, filed on Apr. 12, 2020.

(51) Int. Cl. A62B 18/02; A62B 18/08; A62B 27/00

(52) U.S. Cl. CPC A62B 18/025; A62B 27/00; A62B 18/08

(57) ABSTRACT

An apparatus for identifying leak of a worn face mask, including a leak detection material configured to detect the leakage location according to the seal between a face mask and a wearer's face.

### CROSS REFERENCE TO RELATED APPLICATION

This application claims the benefit of filing date of U.S. Provisional Application Ser. No. 63/008,817, entitled “AN APPARATUS FOR IDENTIFYING LEAKS OF FACE MASK” filed Apr. 12, 2020 under 35 USC § 119(e)(1).

### BACKGROUND OF THE INVENTION

### 1. Field of the Invention

The present invention relates to an apparatus for identifying leak of a 15 worn face mask, which also relates to a face mask fit test or fit check, and more particularly, to an apparatus for identifying the leak location of the face mask.

### 2. Description of Related Art

Exposure to high concentration of air pollution air can result in cardiovascular or respiratory disease. The pollution sources can be from automotive emissions, industrial emissions, and surgical smoke generated during surgery. For medical personnel who are exposed to high concentration of surgical smoke, using proper protective equipment is 25 needed.

Face mask includes tight-fitting face mask and loose-fitting face mask. Respirators usually refer to “tight-fitting” face masks, such as filtering facepiece respirator (such as N95 respirator), Powered Air-Purifying Respirator (PAPR), Supplied-Air Respirator (SR) or Self-Contained Breathing Apparatus (SCBA). Respirator can help protect the users exposed in the hazardous environment. Loose-fitting face mask includes surgical mask, dust mask, and cloth face covering. Loose-fitting face mask are more comfortable to wear, but provides less protection to the workers.

Both tight-fitting face mask and loose-fitting face mask are capable of protecting the wearers to some extent. For different environments, users can choose different kinds of face masks. Note that in a hazardous environment, even a little leak can pose danger to the wearer. Thus, identifying the face mask leak location is important and can help protect the user wearing the face mask.

People who wear respirators need to perform fit test and fit check. However, according to a research on Journal of Hospital Infection by J. L. Derrick et al (Derrick J L, Chan Y F, Gomersall C D, Lai S F. Predictive value of the user seal check in determining half-face respirator fit. J Hosp. Infect. 2005; 59, 152-155), the existing user fit (seal) check is inaccurate. In addition, the face mask users have to perform a fit check every time they put the face mask on. This is time consuming and often cannot immediately identify the location of the leak.

In US Pat. No. 2016/0166859 A1, Rachapudi et al disclosed a more accurate method. The method used a pressure sensor and a computing device to quantify the leak. Although this may be a more accurate method, the additional electronic device may not be economically feasible to those with a tight budget. In addition, this method cannot find the location of the leak. In CN 1919376 B, Zhu disclosed a kind of low-cost leak detection device which is capable of locating the leak location. This device does not need a pressure sensor, but it requires a transparent material for one side and requires fine frosted texture for the other side. However, this leak detection device cannot be applied to the commercial N95 respirators or surgical masks.

Therefore, it is desirable to provide an improved apparatus for identifying leak location of face mask and can be applied to commercial face mask or respirators (e.g., N95, N99, N100 respirators).

### SUMMARY OF THE INVENTION

In this regard, the first aspect of the present invention is to provide an apparatus for identifying leak of face mask comprising:

an inner layer, adapted to be close to a wearer's face; and

an outer layer, located on an opposite side of the inner layer, an inner space being between the inner layer and the outer layer, the inner space comprising at least one filling material as a leak detection material.

Optionally or preferably, in the apparatus for identifying leak of face mask, the various filling materials are pressure sensitive material, pressure indicating sensor film, pressure sensitive color gel envelope, pressure sensitive gel envelope, pressure sensitive color ball envelope, pressure sensitive ball envelope, temperature sensitive material, thermo-chromatic material, temperature sensitive dye, temperature sensitive ink, temperature sensitive paint, heat-shrink tubing, pressure shrinkable tubing, relative humidity sensitive material, relative humidity indicator paper, scent sensitive material, tape, glue, adhesives, or any combination thereof.

Optionally or preferably, the apparatus for identifying leak of face mask further comprising an interface pad, provided in between the outer layer and the face mask, the material of the interface pad is cotton, plastic, fabric, woven material, non-woven fabric, natural fiber, artificial fiber, or any combination thereof.

Optionally or preferably, in the apparatus for identifying leak of face mask, the inner space comprises:

a first portion, filled with the at least filling material, a side of the first portion adapted to be connected to a face mask; and

a second portion, being adjacent to the other side of the first portion;

wherein the first portion and the second portion are partitioned by a partition material, the partition material is a permeable material.

Optionally or preferably, in the apparatus for identifying leak of face mask, the at least one filling materials filled in the first portion move to the second portion by the variations of pressure, temperature, relative humidity, and/or scent, wherein pressure, temperature, relative humidity, and/or scent can be used to detect leaks.

Optionally or preferably, in the apparatus for identifying leak of face mask, the first portion comprises a plurality of first sections, and the second portion comprises a plurality of second sections, the first sections of the first portion being filled with the respective filling materials, the second sections of the second portion corresponding to the first sections, respectively.

Optionally or preferably, in the apparatus for identifying leak of face mask, the first sections of the first portion and the second sections of the second portion are divided into extremely fine sections, each having a volume between 0.1 mm³ and 1000 mm³ to precisely indicate the leak location.

Optionally or preferably, in the apparatus for identifying leak of face mask, the side of the first portion is re-connectable to the face mask after being disconnected from the face mask.

Optionally or preferably, in the apparatus for identifying leak of face mask, the first portion surrounds to the face mask.

Optionally or preferably, in the apparatus for identifying leak of face mask, the partition material is a porous membrane.

Optionally or preferably, in the apparatus for identifying leak of face mask, the first portion and the second portion are transparent air envelopes.

Optionally or preferably, in the apparatus for identifying leak of face mask, the face mask is a loose-fitting face mask, the loose-fitting face mask comprising a surgical mask, a dust mask, or a cloth mask.

Optionally or preferably, in the apparatus for identifying leak of face mask, the face mask is a tight-fitting face mask, the tight-fitting face mask comprising a N95, N99, N100, P95, P99, P100, R95, R99, R100 respirator, an Air-purifying Respirator, a Powered Air-Purifying Respirator (PAPR), a Supplied-Air Respirator (SR) or a Self-Contained Breathing Apparatus (SCBA).

The first aspect of the present invention further provides a method for identifying leak of face mask, executed by the apparatus for identifying leak of face mask in the first aspect, the method comprising:

Step S11: connecting the apparatus for identifying leak of face mask to the face mask;

Step S12: putting the face mask on a wearer until attaching to the wearer's face;

Step T11: determining if the second portion is thoroughly filled with the filling materials from the first portion; if the determination result is yes, it is a no-leak condition; if the determination result is no, it is a leak condition, and then proceed to step T12;

Step T12: identifying an unfilled position as the leak location.

Optionally or preferably, in the method for identifying leak of face mask, the determination in Step T11 and/or Step T12 is performed by eyes.

Optionally or preferably, in the method for identifying leak of face mask, the determination in Step T11 and/or Step T12 is performed by scanning the wearer's face through an image detector.

In particular, the one or more optional features in the first aspect may be applied to the apparatus for identifying leak of face mask in the second aspect or the third aspect, individually or in combination, if needed.

A second aspect of the present invention is to provide an apparatus for identifying leak of face mask, comprising:

an inner layer, adapted to be close to a wearer's face; and

an outer layer, located on an opposite side of the inner layer, an inner space being between the inner layer and the outer layer, the inner space comprising:
a first portion, filled with at least one filling material, one side of the first portion being adapted to be connected to a face mask; and
a second portion, being adjacent to another side of the first portion, an interior of the first portion provided with a reservoir, constructed to store the at least one filling materials, wherein the inner space is designed such that material is not interchangeable between the first portion and the second portion.

Optionally or preferably, in the apparatus for identifying leak of face mask, the at least one filling material in the reservoir moves outward using capillary phenomenon.

The second aspect of the present invention further provides a method for identifying the leak of the face mask, executed by the apparatus for identifying leak of face mask of the second aspect, the method comprising:

Step S21: connecting the apparatus for identifying leak of face mask to the face mask;

Step S22: putting the face mask on a wearer's face until attaching to the wearer's face;

Step T21: determining if the second portion is thoroughly filled with the filling materials; if the determination result is yes, it is a no-leak condition; if the determination result is no, it is a leak condition, and then proceed to step T12;

Step T22: identifying an unfilled position as the leak location.

Optionally or preferably, in the method for identifying leak of face mask, the determination in Step T21 and/or Step T22 is performed by eyes.

Optionally or preferably, in the method for identifying leak of face mask, the determination in Step T21 and/or Step T22 is performed by scanning the wearer's face through an image detector.

A third aspect of the present invention is to provide an apparatus for identifying leak of face mask comprising:

an inner layer, adapted to be close to a wearer's face; and

an outer layer, located on an opposite side of the inner layer, an inner space being between the inner layer and the outer layer, the inner space comprising:
at least one first portion, filled with the at least one filling material, one side of the first portion being adapted to be connected to a face mask; and
at least one second portion, being adjacent to another side of the first portion;

wherein the at least one first portion and the at least one second portion are partitioned by a partition material, and the partition material is a permeable material.

Optionally or preferably, in the apparatus for identifying leak of face mask, area of each second portion is equal to or smaller than area of each first portion.

The third aspect of the present invention further provides a method for identifying the leak of the face mask, executed by the apparatus for identifying leak of face mask of the third aspect, the method comprising:

Step S31: connecting the apparatus 10 for identifying leak of face mask to the face mask;

Step S32: putting the face mask on a wearer's face until attaching to the wearer's face;

Step T31: determining if the second portion is thoroughly filled with the filling materials from the first portion; if the determination result is yes, it is a no-leak condition; if the determination result is no, it is a leak condition, and proceed to step T32;

Step T32: identifying an unfilled position as the leak location.

Optionally or preferably, in the method for identifying leak of face mask, the determination in Step T31 and/or Step T32 is performed by eyes.

Optionally or preferably, in the method for identifying leak of face mask, the determination in Step T31 and/or Step T32 is performed by scanning the wearer's face through an image detector.

In the aforementioned first aspect, second aspect, and third aspect, in the first aspect, the inner space of the apparatus may include at least one filling material, the inner space may be an undivided space, or the inner space may be further divided into a first portion and a second portion. The first portion and the second portion are partitioned by a partition material; in the second aspect, the first portion and the second portion of the apparatus are partitioned and they are not material-interchangeable; in the third aspect, the apparatus has at least one first portion and at least one second portion, wherein the at least one first portion and the at least one second portion are partitioned by a partition material, and the partition material can be a permeable material.

Other objects, advantages, and novel features of the invention will become more apparent from the following detailed description when taken in conjunction with the accompanying drawings.

### BRIEF DESCRIPTION OF THE DRAWINGS

FIG. 1A is a schematic diagram of the interior of the apparatus for identifying leak of face mask and the face mask, according to an embodiment of the present invention;

FIG. 1B is a schematic diagram of the exterior of the apparatus for identifying leak of face mask and the face mask, according to an embodiment of the present invention;

FIG. 2A is a schematic diagram of the apparatus for identifying leak of face mask, according to an embodiment of the first aspect of the present invention;

FIG. 2B is a schematic diagram of the apparatus for identifying leak of face mask, according to another embodiment of the first aspect of the present invention;

FIG. 2C is a schematic diagram of the apparatus for identifying leak of face mask, according to still another embodiment of the first aspect of the present invention;

FIG. 3A is a schematic diagram of an arrangement of the apparatus for identifying leak of face mask, according to the second aspect of the present invention;

FIG. 3B is a schematic diagram of another arrangement of the apparatus for identifying leak of face mask, according to the second aspect of the present invention;

FIG. 4 is a schematic diagram of the apparatus for identifying leak of face mask, according to the third aspect of the present invention;

FIG. 5A is a schematic diagram of the apparatus for identifying leak of face mask, which illustrates that the apparatus is combined with an interface pad; and

FIG. 5B is a schematic diagram of the apparatus for identifying leak of face mask, which illustrates that the apparatus is combined with another interface pad.

### DETAILED DESCRIPTION OF THE EMBODIMENT

Different embodiments of the present invention are provided in the following description. These embodiments are meant to explain the technical content of the present invention, but not meant to limit the scope of the present invention. A feature described in an embodiment may be applied to other embodiments by suitable modification, substitution, combination, or separation.

It should be noted that, in the present specification, when a component is described to have an element, it means that the component may have one or more of the elements, and it does not mean that the component has only one of the elements, except otherwise specified.

Moreover, in the present specification, the ordinal numbers, such as “first” or “second”, are used to distinguish a plurality of elements having the same name, and it does not mean that there is essentially a level, a rank, an executing order, or a manufacturing order among the elements, except otherwise specified. A “first” element and a “second” element may exist together in the same component, or alternatively, they may exist in different components, respectively. The existence of an element described by a greater ordinal number does not essentially means the existent of another element described by a smaller ordinal number.

In the present specification, the so-called feature A “or” or “and/or” feature B means A exists solely, B exists solely, or A and B exist at the same time; the so-called feature A “and” feature B means A and B exist at the same time; the so-called “include”, “comprise”, “have”, or “contain” refers to include, but are not limited thereto, except otherwise specified.

Moreover, in the present specification, the terms, such as “top”, “bottom”, “left”, “right”, “front”, “back”, or “middle”, as well as the terms, such as “on”, “above”, “under”, “below”, or “between”, are used to describe the relative positions among a plurality of elements, and the described relative positions may be interpreted to include their translation, rotation, or reflection.

Moreover, in the present specification, when an element is described to be arranged “on” another element, it does not essentially mean that the elements contact the other element, except otherwise specified. Such interpretation is applied to other cases similar to the case of “on”.

Moreover, in the present specification, the terms, such as “preferably” or “advantageously”, are used to describe an optional or additional element or feature, and in other words, the element or the feature is not an essential element, and may be ignored in some embodiments.

Moreover, in the present specification, when an element is described to be “suitable for” or “adapted to” another element, the other element is an example or a reference helpful in imagination of properties or applications of the element, and the other element is not to be considered to form a part of a claimed subject matter; similarly, except otherwise specified; similarly, in the present specification, when an element is described to be “suitable for” or “adapted to” a configuration or an action, the description is made to focus on properties or applications of the element, and it does not essentially mean that the configuration has been set or the action has been performed, except otherwise specified.

Moreover, in the present specification, a value may be interpreted to cover a range within ±10% of the value, and in particular, a range within ±5% of the value, except otherwise specified; a range may be interpreted to be composed of a plurality of subranges defined by a smaller endpoint, a smaller quartile, a median, a greater quartile, and a greater endpoint, except otherwise specified.

Moreover, in the present specification, the term “face mask” may refer to loose-fitting face mask, such as a surgical face mask, a dust mask, or a cloth mask; the term “face mask” may also refer to tight-fitting face mask, such as N95, N99, N100, P95, P99, P100, R95, R99, R100 respirator, Air-purifying Respirator, Powered Air-Purifying Respirator (PAPR), Supplied-Air Respirator (SR) or Self-Contained Breathing Apparatus (SCBA). The term “interior of face mask” may refer to the space between the face mask and the mouth or the nose of the wearer; the term “exterior of face mask” may refer to the ambient condition of the wearer. The term “leak” of face mask may refer to the “unfitted” wearing of the face mask; the term face mask “no leak” may refer to the “fitted” wearing of the face mask.

As the most basic embodiment, the apparatus 10 for identifying leak of face mask of the present invention may be a pressure-changing cloth or material, which is attached or embedded to the face mask. The pressure-changing cloth may be circular shaped pasted around the circumference of the face mask. Alternatively, the pressure-changing cloth can be a cloth that overlaps the entire face mask (its area should be slightly larger than the face mask so that the leak detection material (i.e., pressure-changing cloth) of the face mask and can be observed by the user or other people or machine) and immediately locate the leak location.

In the most basic embodiment, when the user put the face mask on, the contact pressure between the mask and the wearer's face will induce the pressure-changing cloth to change color. According to the discoloration principle of the pressure-changing cloth, if the mask and the wearer's face are tight without leakage, all areas of the pressure-changing cloth will change colors; if the two are not tight and leaking, some areas of the pressure-changing cloth will not change colors, indicating the location of the leak. Of course, it is possible to make a design where the above principle is reversed, that is, a location that changes colors refers to leakage, and a area that does not change colors refers to no leakage.

(First Aspect)

(Apparatus Structure)

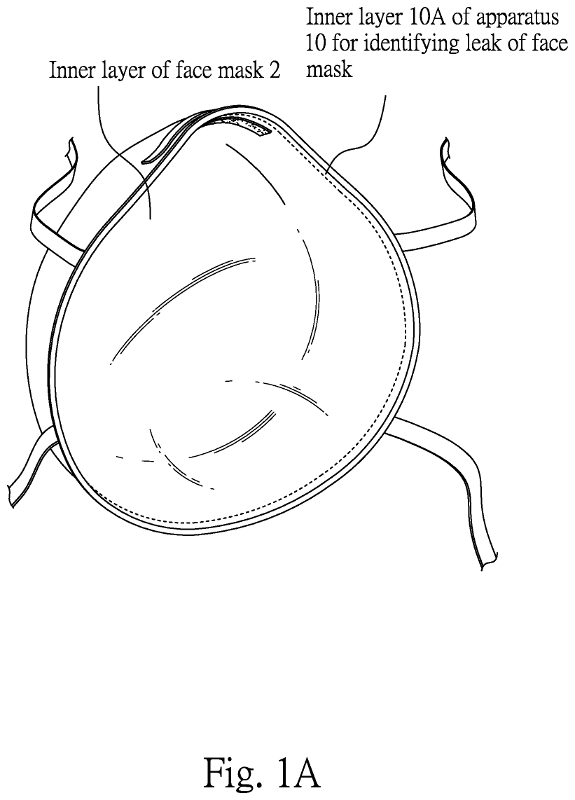

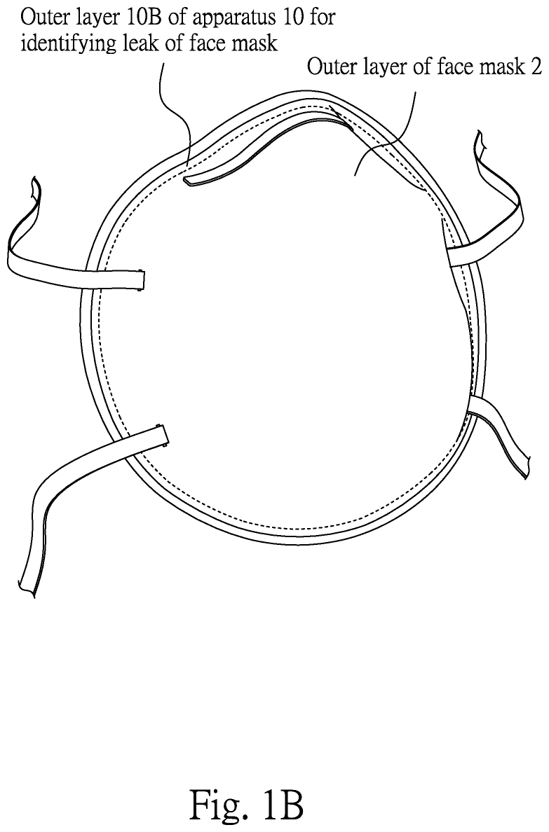

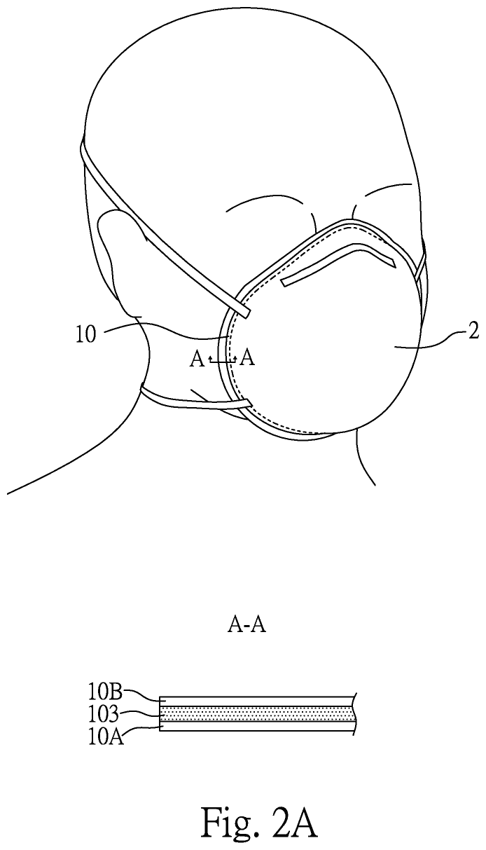

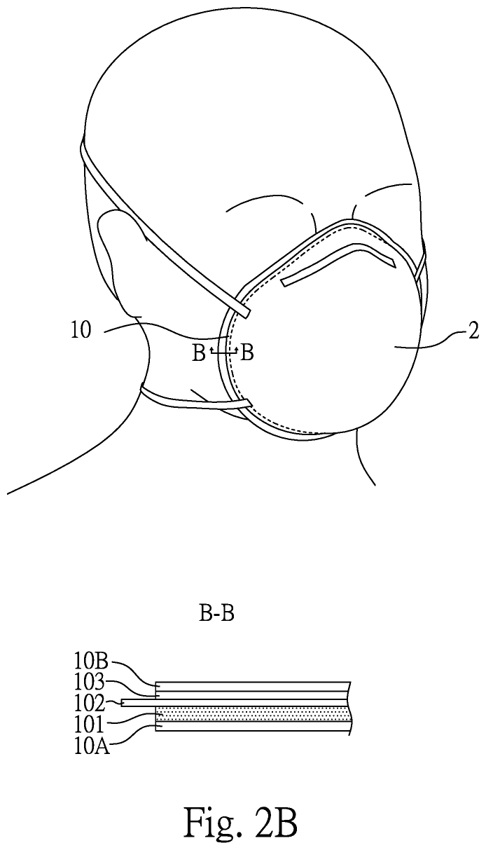

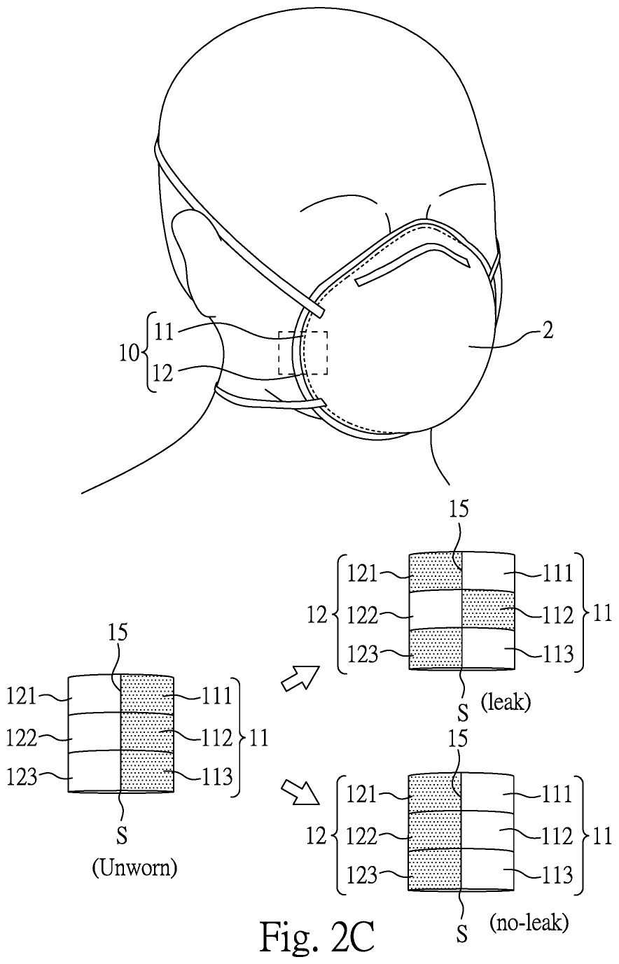

FIG. 1A is a schematic diagram of the interior of the apparatus 10 for identifying leak of face mask and the face mask 2, according to an embodiment of the present invention. FIG. 1B is a schematic diagram of the exterior of the apparatus 10 for identifying leak of face mask and the face mask 2, according to an embodiment of the present invention. FIG. 2A is a schematic diagram of the apparatus 10 for identifying leak of face mask, according to an embodiment of the first aspect of the present invention. FIG. 2B is a schematic diagram of the apparatus 10 for identifying leak of face mask, according to another embodiment of the first aspect of the present invention. FIG. 2C is a schematic diagram of the apparatus 10 for identifying leak of face mask, according to still another embodiment of the first aspect of the present invention.

In one embodiment of the first aspect, the apparatus 10 for identifying leak of face mask has an inner layer 10A (as shown in FIG. 1A, the view observing from the interior of the face mask) and an outer layer 10B (as shown in FIG. 1B, the view observing from the exterior of the face mask). The inner layer 10A is suitable for being close to a wearer's face, and the outer layer 10B is on the opposite side of the inner layer 10A. Between the inner layer 10A and the outer layer 10B is an inner space S. The inner space S can be filled with at least one filling material as a leak detection material. In one embodiment, the filling material may be a color-changing layer 103.

In another embodiment of the first aspect, the filling materials in the inner space S are materials with sandwich structure formed by a pressure sensitive layer 101, an isolating layer 102, and a color-changing layer 103. After the face mask 2 is worn, the isolating layer 102 should be removed, so that the pressure sensitive layer 101 and the color-changing layer 103 can contact each other, and change the color on the color-changing layer 103. The pressure-changing material can surrounds the face mask, when the user wears the face mask, if a leak persists, the pressure-changing material will change color at the location of the leak. On the other hand, is there is no leak, the pressure-changing material will maintain the same color showing there is no leak.

In still another embodiment of the first aspect, the inner space S includes a first portion 11 and a second portion 12. The first portion 11 can be filled with at least one filling material. One side of the first portion is suitable for being connected with a face mask 2. More specifically, the apparatus 10 for identifying leak of face mask can be connected with the face mask 2. It can be disconnected from the face mask 2 after the connection, and then be reconnected (i.e., a reusable apparatus). The second portion 12 is adjacent to the other side of the first portion 11, and the first portion 11 and the second portion 12 are partitioned by a partition material 15. The partition material 15 is a permeable material.

In an embodiment, one of the inner layer 10A or the outer layer 10B can integrated with the face mask 2 to one piece. Additionally, it can be understood that one of the inner layer 10A or the outer layer 10B is the face mask 2. In this case, the first aspect, the apparatus 10 for identifying leak of face mask cannot be separated from the face mask 2, but can be an integrated face mask product.

In the first aspect, the first portion 11 and the second portion 12 may each include a plurality of first sections 111, 112, 113, and a plurality of second sections 121, 122, 123, wherein the first sections 111, 112, 113 are filled with respective filling materials. The second sections 121, 122, 123 are on the other side of the first sections 111, 112, 113, respectively. In addition, the first sections 111, 112, 113 and the second sections 121, 122, 123 are divided by the partition materials 15 (i.e., the first sections 111, 112, 113 and the second sections 111, 112, 123 are the parts divided from the first portion 11 and the second portion 12.)

Furthermore, the first sections 111, 112, 113 of the first portion 11 and the second sections 121, 122, 123 of the second portion 12 may be further divided into extremely fine sections, wherein the volume of each extremely fine sections may be in a range from 0.1 mm³ to 1000 mm³, so as to identify the leak location in more accurate way. The range of the volume mentioned above is exemplary, and the volume can be designed in range of any value, according to the actual accuracy demand.

(Materials)

In the first aspect, the partition material 15 may be porous membrane, but not limited thereto. The porous membrane can be a permeable material that can be used to detect leaks in the face mask. The face mask leak detection principle will be explained below. The filling materials may be pressure sensitive material, pressure indicating sensor film, pressure sensitive color gel envelope, pressure sensitive gel envelope, pressure sensitive color ball envelope, pressure sensitive ball envelope, temperature sensitive material, thermo-chromatic material, temperature sensitive dye, temperature sensitive ink, temperature sensitive paint, heat-shrink tubing, pressure shrinkable tubing, relative humidity sensitive material, relative humidity indicator paper, scent sensitive material, tape, glue, adhesives, or any combination thereof, but not limited thereto. Any kinds of filling materials that can be detected by eyes or by devices such as image detector can be adapted.

(Principle of Leak Identification)

With reference to the example illustrated in FIG. 2A, the apparatus 10 for identifying leak of face mask is filled with a color-changing layer 103. The color-changing layer 103 may be a pressure sensitive material. When the face mask 2 is being worn, pressure is being applied to the face mask 2. The color of the pressure sensitive material can change under different pressure.

With reference to example illustrated in FIG. 2B, a sandwich structure is formed by a pressure sensitive layer 101, an isolating layer 02, and a color-changing layer 103 in the inner space S of apparatus 10 for identifying leak of face mask. After the face mask 2 is worn, the wearer can draw out the isolating layer 102 to let the pressure sensitive layer 101 and the color-changing layer 103 contact each other and change the color on the color-changing layer 103. Adding an isolating layer 102 between the pressure sensitive layer 101 and the color-changing layer 103 can isolate them from contacting each other during irrelevant action period (such as transportation), thus avoiding any error of sensing.

With reference to the example illustrated in FIG. 2C, the first sections 111, 112, 113 of the first portion 11 are filled with at least one filling material (indicated by printing dots), as described above. Herein we use the pressure sensitive color gel envelope as the filling material for example to explain one embodiment. When the face mask 2 is not worn, the pressure sensitive color gel envelopes are filled in the first sections 111, 112, 113 of the first portion 11; when the face mask 2 is being worn, because pressure is applied on the face mask 2, the pressure sensitive color gel envelopes will pass through the partition material 15 (such as porous membrane) and start to move to the second sections 121, 122, 123 of the second portion 12. After being worn, in a no-leak (or fitted) condition, the pressure sensitive color gel envelope favorably moves to the second sections 121, 122, 123 of the second portion 12; in a leak (or unfitted) condition, some pressure sensitive color gel envelopes will not be able to move through the partition material 15 and move to the second sections 121, 122, 123 of the second portion 12, but stay in the first sections 111, 112, 113, instead. The positions in the second sections without the materials are exactly the leak locations. Accordingly, the first aspect of the present invention can determine if a leak of a face mask 2 occurs and then identify the leak location by seeing that if the pressure sensitive color gel envelopes move to the designated positions.

Still with reference to the example of FIG. 2C, when there is no leaks, all the pressure sensitive color gel envelopes pass through the partition materials 15 and move to the second sections 121, 122, 123. However, when a leak occurs, in one certain embodiment, the pressure sensitive color gel envelope in the first section 112 is not able to move to the second section 122, but stay in the first section 112, and the second section 122 indicates the leak location.

Similarly, if the filling material is the temperature sensitive material, when the face mask 2 is worn, the temperature in the interior of the face mask is usually high, the temperature in the exterior of the face mask is usually low; therefore, a temperature variation (or temperature difference) occurs. When the temperature sensitive material detects the temperature variation, the temperature sensitive material will pass through the partition material 15 and move to the second sections 121, 122, 123 of the second portion 12; however, if a leak occurs, the cold air in the exterior of the face mask may flow into the interior of the face mask, as a result, the temperature variation is insufficient to force all the temperature sensitive materials pass through the partition material 15 and move to the second sections 121, 122, 123 of the second portion 12, that is, the sections without the materials moving from the first sections are exactly the sections have leaks.

Relative humidity sensitive material moves, through detecting the relative humidity variation between the interior and the exterior of the face mask 2; scent sensitive material moves, through detecting the scent variation between the interior and the exterior of the face mask 2. The aforementioned physical terms such as pressure, temperature, relative humidity, and scent are merely exemplary but not limited thereto.

(Optional Features)

The permeable partition material 15 is preferably a “bidirectional” material, that is, after the mask 2 is used, the filling material can return to the first portion 11 again. Based on the consideration of different material properties, the partition material 15 can also be a unidirectional material, that is, the filling material cannot return to the first part 11 after the move.

In the conditions of filling with “color” gel envelope, gel, ball envelope or ink, the first portion 11 and the second portion 12 themselves, preferably, may be transparent air envelopes. As such, the leak locations can be clearly identified by eyes.

In above arrangements of the first aspect, one side of the first portion 11 of the apparatus 10 for identifying leak of face mask may wholly surround to the face mask 2. It is interesting to note that, the apparatus 10 may also be partially connected to the face mask 2 (not wholly surround to), for example, merely attached to the nose bridge or to the jaw, to reduce the cost and the space.

In the above embodiments, the apparatus 10 for identifying leak of face mask has been connected to the face mask 2. But it should be noted that, the apparatus 10 may be embedded into the face mask 2, being integral with the face mask 2; alternatively, it may be a standalone apparatus (i.e., face mask's color pad), and attached to the face mask 2 while using. Thus, the size and length of the apparatus 10 for identifying leak of face mask is adjustable, or even customized to match different face masks. Thus, they are compatible with commercial face masks. In addition, the plurality of sensitive materials are solutions and can be directly filled into the first portion 11. Therefore, the process is simple and the cost is low.

(Method)

A yet embodiment of the present invention provides a method for identifying leak of face mask, and it can be accomplished by the apparatus for identifying leak of face mask in the embodiment of the first aspect. The method includes the steps as follows:

Step S11: connecting the apparatus 10 for identifying leak of face mask to the face mask 2;

Step S12: putting the face mask 2 on the wearer's face until attaching to the wearer's face;

Step T11: determining if the second portion 1 is thoroughly filled with the filling materials from the first portion 11; if the determination result is yes, it is a no-leak condition; if the determination result is no, it is a leak condition, and then proceed to step T12;

Step T12: identifying an unfilled position, and that position is the leak location.

In one embodiment, the determination in Step T11 and/or Step T12 is performed by eyes, while, in another embodiment, the determination in Step T11 and/or Step T12 is performed by scanning the wearer's face through an image detector. The image detector can be disposed at the entrance of the operating room, premises, or any kinds of rooms. When the wearers walk into the door, the detector can immediately detect and determine whether the face mask leaks or not, so that operations, works or evens can be proceeding safely.

(Second Aspect)

(Apparatus Structure)

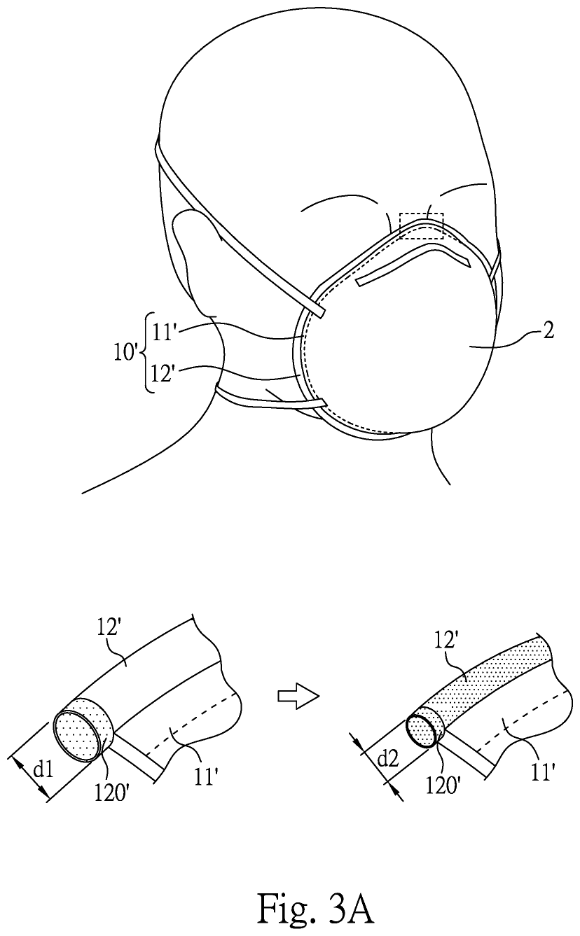

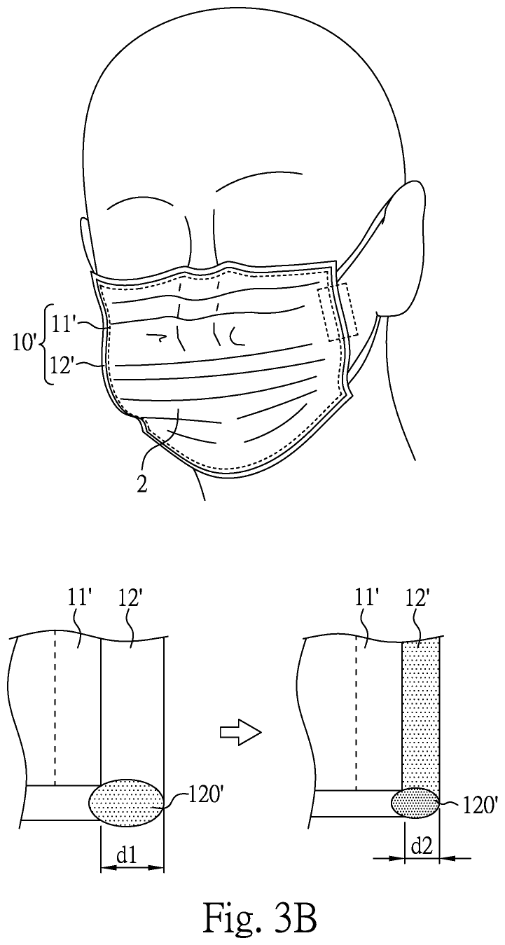

FIG. 3A is a schematic diagram of an arrangement of the apparatus 10′ for identifying leak of face mask, according to the second aspect of the present invention. FIG. 3B is a schematic diagram of another arrangement of the apparatus 10′ for identifying leak of face mask, according to the second aspect of the present invention.

In the second aspect, the apparatus 10′ for identifying leak of face mask has an inner layer 10A (as shown in FIG. 1A) and an outer layer 10B (as shown in FIG. 1B). The inner layer 10A is suitable for being close to a wearer's face, and the outer layer 10B is on the opposite side of the inner layer 10A. Between the inner layer 10A and the outer layer 10B is an inner space S. The inner space S includes a first portion 11′ and a second portion 12′. One side of the first portion 11′ is suitable for being connected with a face mask 2. More specifically, the apparatus 10′ for identifying leak of face mask can be connected with the face mask 2. It can be disconnected from the face mask 2 after the connection, and then be reconnected (i.e., a reusable apparatus). The second portion 12′ is adjacent to the other side of the first portion 11′. It should be noted that the first portion 11′ and the second portion 12′ are partitioned.

In the second aspect, the apparatus 10′ for identifying leak of face mask may further include a reservoir 120′. The reservoir 120′ may be disposed somewhere in the interior of the second portion 12′. The reservoir 120′ is constructed to store at least one filling material.

(Materials)

In the second aspect, the filling materials shown in the first aspect may be applied to the embodiments of the second aspect as well. Since the second aspect applies the capillary phenomenon, the second portion preferably is a heat-shrink tubing. The heat-shrink tubing will shrink during heating when the user put the face mask on. Preferably, the filling materials are color liquid, such as paint or color gel. The principle of the leak identification will be explained below.

(Principle of Leak Identification)

In FIG. 3A and FIG. 3B, at least one filling material (indicated by printing dots) is filled in the reservoir 120′ of the second portion 12′, the filling material preferably being the paint or color gel. Herein we use the color gel as the filling material for example to explain one embodiment. The second aspect (FIG. 3A, FIG. 3B) is different from the first aspect (FIG. 2) since the first portion 11′ and the second portion 12′ of the apparatus 10′ for identifying leak of face mask are not divided into a plurality of first sections and a plurality of second sections. The diameter of the heat shrink tube of the second portion 12′ will change. When the face mask 2 is not worn, the diameter of the second portion 12′ of the heat shrink tubing is a first diameter d1, and at this moment, the filling material is filled in the reservoir 120′. After the face mask 2 is worn, the wearer's body temperature heats the heat-shrink tubing and the first diameter d1 change to a second diameter d2, wherein by the heat-shrinking effect, the second diameter d2 is equal to or smaller than the first diameter d1. When there is no leak in the face mask, the body temperature changes the diameter of the heat shrink tubing, enabling the second diameter d2 to be smaller than a critical diameter, so the color gel in the reservoir 120′ can move. When there is a leak, there will not be enough heat provided by the contact between the face and gas mask. Therefore, the second diameter d2 at the leak location within the second portion 12′ cannot change to be smaller than the critical diameter, so the capillary phenomenon cannot be triggered, and the color gel in the reservoir 120′ cannot move. Thus, there are unfilled parts in the second portion 12′ indicating the location of the leak. If we choose to use a thermal expansion tube instead of heat-shrink tubing, a contrast principle can be applied. Technicians can understand the design of thermal expansion tube in nature of the present invention, so the details will not be described herein again. Furthermore, we can also use a pressure shrinkable tubing to replace the heat-shrink tubing. When the user put the mask on and there is no leak, there will be enough pressure to make the diameter of the pressure shrinkable tubing smaller. If a leak occurs or when the user takes the mask off, there will not be enough pressure to make the pressure shrinkable tubing smaller.

(Optional Features)

In the second aspect, one or more features from the first aspect can be applied for the apparatus 10′ for identifying leak of face mask in the second aspect, and thus, the details will not be described herein again.

When a leak occurs, the apparatus 10′ for identifying leak of face mask in the second aspect applies the capillary phenomenon, if the second diameter d2 exceed beyond the critical diameter, the capillary phenomenon will fail to work, and the color gel cannot move past the leak location.

(Method)

Yet another embodiment of the present invention provides a method for identifying the leak of the face mask. It can be accomplished through the apparatus 10′ for identifying leak of face mask in the embodiments of the second aspect. The method includes the steps as follows:

Step S21: connecting the apparatus 10′ for identifying leak of face mask to the face mask 2;

Step S22: putting the face mask 2 on the wearer's face until attaching to the wearer's face;

Step T21: determining if the second portion 12′ is thoroughly filled with the filling material; if the determination result is yes, it is a no-leak condition; if the determination result is no, it is a leak condition, and then proceed to step T12;

Step T22: identifying an unfilled position, and that position is the leak location.

In one embodiment, the determination in Step T21 and/or Step T22 is performed by eyes, while, in another embodiment, the determination in Step T21 and/or Step T22 is performed by scanning the wearer's face through an image detector.

(Third Aspect)

(Apparatus Structure)

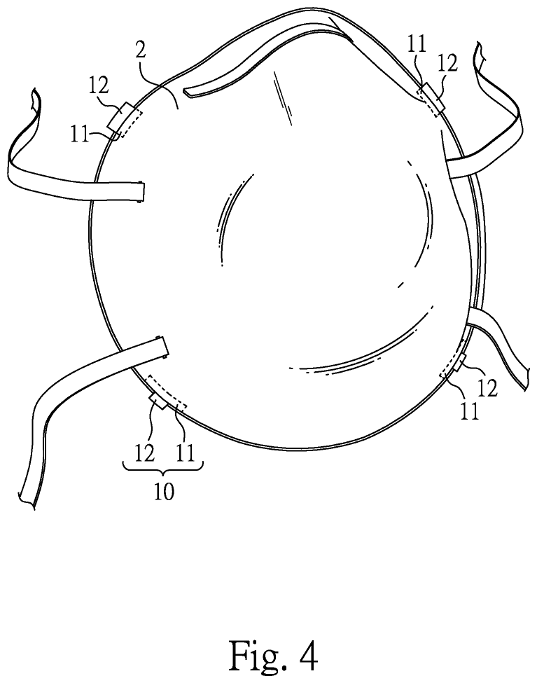

FIG. 4 is a schematic diagram of the apparatus 10 for identifying leak of face mask, according to the third aspect of the present invention.

In the third aspect, the apparatus 10 for identifying leak of face mask has an inner layer 10A (as shown in FIG. 1A) and an outer layer 10B (as shown in FIG. 1B). The inner layer 10A is suitable for being close to a wearer's face, and the outer layer 10B is on the opposite side of the inner layer 10A. Between the inner layer 10A and the outer layer 10B is an inner space S. The inner space S includes a first portion 11 and a second portion 12. The first portion 11 can be filled with at least one filling material. One side of the first portion is suitable for being connected with a face mask 2. More specifically, the apparatus 10 for identifying leak of face mask can be connected with the face mask 2. It can be disconnected from the face mask 2 after the connection, and then reconnected (i.e., a reusable apparatus). The second portion 12 is adjacent to the other side of the first portion, and the first portion 11 and the second portion 12 are partitioned by a partition material 15. The partition material is a permeable material.

In the third aspect, the apparatus 10 for identifying leak of face mask includes at least one first portion, filled with at least one filling material (indicated by printing dots). The first portion 11 is suitable for partially or completely covering the face mask 2. Each second portion 12 is adjacent to each first portion correspondingly, and sticks out or extends over the face mask 2. In this embodiment, the area of each second portion 12 is equal to or smaller than that of each first portion 11. Each first portion 11 and each second portion 12 are partitioned by partition materials 15 (as shown in FIG. 2).

(Materials)

In the third aspect, the filling materials shown in the first aspect may be applied in the embodiments of the third aspect as well, and the details will not be described herein again.

(Principle of Leak Identification)

In FIG. 4, all the plurality of the first portions 11 are filled with at least one filling materials, as mentioned above. They may be the same material, or different materials. Herein we use the pressure sensitive color gel envelope as the filling material for example to explain one embodiment. When the face mask 2 is not worn, the pressure sensitive color gel envelopes are filled in the first portion 11; when the face mask 2 is worn, because a pressure is applied to the face mask 2, the pressure sensitive color gel envelopes will pass through the partition material 15 (such as porous membrane) and move to the second portion 12. After the face mask is worn, in a no-leak condition (also called fitted), the pressure sensitive color gel envelopes completely move to the second portion; in a leak (also called unfitted) condition, some pressure sensitive color gel envelopes are not able to pass through the partition material 15 to move to the second portion 12, but still stay in the first portion 11. The positions of the second portions 12 where those pressure sensitive color gel envelopes cannot move to are exactly the leak locations.

Similarly, in the condition of filling with different sensitive materials, when the face mask 2 is worn, those sensitive materials will pass through the partition materials 15 (such as porous membrane) and move to the second portion 12, by detecting the variation of the physical quantity. Those physical terms, such as pressure, temperature, relative humidity, are exemplary but not limited thereto.

(Optional Features)

In the third aspect, one or more features from the first aspect can be applied for the apparatus 10 for identifying leak of face mask in the third aspect.

Here, it should be noted that, in addition to the proper wear, people's face shape or face feature is not symmetrical, more or less. Probably, the asymmetrical part is the reason of the leak or the unfit of the face mask 2. The apparatus for identifying the leak of the face mask 10 provided in the third aspect can be a standalone apparatus. In the meanwhile, it has the plurality of first portions 11 and the plurality of second portions 12, and it can be re-attached to the face mask 2 after being disconnected, so that the apparatus is advantageous to find the most possible (or often) leak location. Thus, the apparatus 10 for identifying leak of face mask can be disposed in the position where the leak most likely occurs. As such, it is possible to utilize the apparatus of the third aspect to accomplish the purpose of identifying the leak of the face mask in low-cost and effective ways.

(Method)

A yet embodiment of the present invention provides a method for identifying the leak of the face mask, and it can be accomplished through the apparatus 10 for identifying leak of face mask in the embodiments of the third aspect. The method includes the steps as follows:

Step S31: connecting the apparatus 10 for identifying leak of face mask to the face mask 2;

Step S32: putting the face mask 2 on the wearer's face until attaching to the wearer's face;

Step T31: determining if each of the second portion 12 is thoroughly filled with the filling materials from the first portion 11; if the determination result is yes, it is a no-leak condition; if the determination result is no, it is a leak condition, and proceed to step T32;

Step T32: identifying an unfilled position, and that position is the leak location.

In one embodiment, the determination of Step T31 and/or Step T32 is performed by eyes, while, in another embodiment, the determination of Step T31 and/or Step T32 is performed by scanning the wearer's face through an image detector.

(Interface Pad of Face Mask)

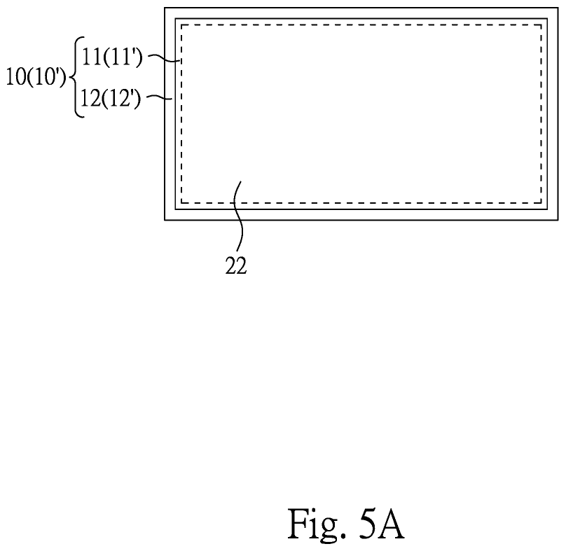

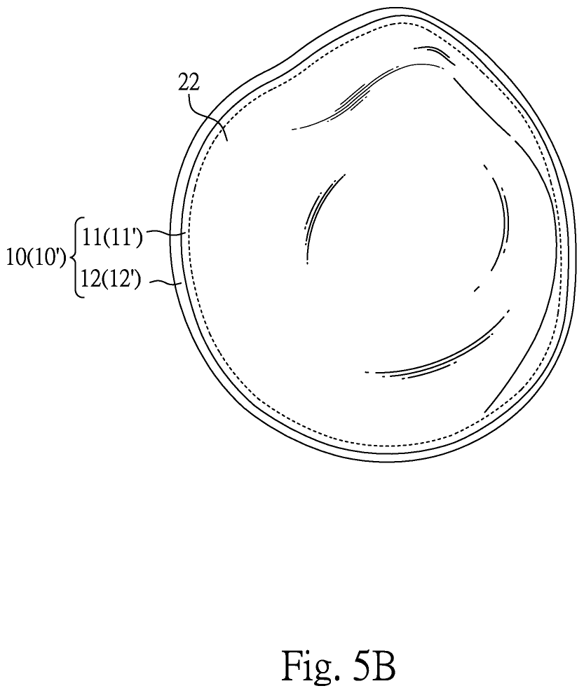

FIG. 5A is a schematic diagram of the apparatus 10 for identifying leak of face mask, which illustrates that the apparatus 10 is combined with an interface pad 22. FIG. 5B is a schematic diagram of the apparatus 10 for identifying leak of face mask, which illustrates that the apparatus 10 is combined with another interface pad 22.

In one embodiment, the apparatus 10 for identifying leak of face mask can include an interface pad 11. It can be disposed between the outer layer of the first portion 11 and the face mask 2. The material of the interface pad 11 may be cotton, plastic, fabric, woven material, non-woven fabric, natural fiber, artificial fiber, or any combination thereof but not limited thereto.

As mentioned above, the apparatus 10 for identifying leak of face mask can combine with the interface pad 22, and can be disposed between the face mask 2 and the mouth or the nose of the wearer, in the interior of the face mask. The interface pad 22 can be provided as a plane type, to contact the mouth or the nose, as shown in FIG. 5A, or alternatively, it can be provided as 3D type, to attach to the interior of the respirator (such as N95 respirator), as shown in FIG. 5B. If using interface pad 22, the wearer's mouse or the nose of the wearers will not directly contact the face mask, so that if there is a requirement of repeatedly wearing the face mask, only to change the interface pad 22 being fine, thereby prolonging the life time of the face mask 2.

Although the present invention has been explained in relation to its preferred embodiment, it is to be understood that many other possible modifications and variations can be made without departing from the spirit and scope of the invention as hereinafter claimed.

What is claimed is:

1. An apparatus for identifying the leak location of worn face mask includes a leak detection material configured to detect the leakage location according to the seal between a face mask and a wearer's face.

2. The apparatus for identifying leak of face mask of claim 1, wherein the leak detection material is pressure-changing material.

3. The apparatus for identifying leak of face mask of claim 1, further comprising:
an inner layer, adapted to be close to the wearer's face; and
an outer layer, located on an opposite side of the inner layer, an inner space being between the inner layer and the outer layer, the inner space comprising at least one filling material as the leak detection material.

4. The apparatus for identifying leak of face mask of claim 3, wherein the various filling materials are pressure sensitive material, pressure indicating sensor film, pressure sensitive color gel envelope, pressure sensitive gel envelope, pressure sensitive color ball envelope, pressure sensitive ball envelope, temperature sensitive material, thermo-chromatic material, temperature sensitive dye, temperature sensitive ink, temperature sensitive paint, heat-shrink tubing, relative humidity sensitive material, relative humidity indicator paper, scent sensitive material, tape, glue, adhesives, or any combination thereof.

5. The apparatus for identifying leak of face mask of claim 3, further comprising an interface pad, provided in between the outer layer and the face mask, the material of the interface pad is cotton, plastic, fabric, woven material, non-woven fabric, natural fiber, artificial fiber, natural materials, artificial materials, or any combination thereof.

6. The apparatus for identifying leak of face mask of claim 3, wherein the inner space comprises:
a first portion, filled with the at least filling material, a side of the first portion adapted to be connected to the face mask; and
a second portion, being adjacent to the other side of the first portion;
wherein the first portion and the second portion are partitioned by a partition material, the partition material is a permeable material.

7. The apparatus for identifying leak of face mask of claim 6, wherein the at least one filling materials filled in the first portion move to the second portion by the variations of pressure, temperature, relative humidity, and/or scent.

8. The apparatus for identifying leak of face mask of claim 6, wherein the first portion comprises a plurality of first sections, and the second portion comprises a plurality of second sections, the first sections of the first portion being filled with the respective filling materials, the second sections of the second portion corresponding to the first sections, respectively.

9. The apparatus for identifying leak of face mask of claim 8, wherein the first sections of the first portion and the second sections of the second portion are divided into extremely fine sections, each having a volume between 0.1 mm³ and 1000 mm³ to precisely indicate the leak location.

10. The apparatus for identifying leak of face mask of claim 4, wherein the side of the first portion is re-connectable to the face mask after being disconnected from the face mask.

11. The apparatus for identifying leak of face mask of claim 4, wherein the first portion surrounds to the face mask.

12. The apparatus for identifying leak of face mask of claim 4, wherein the partition material is a porous membrane.

13. The apparatus for identifying leak of face mask of claim 4, wherein the first portion and the second portion are transparent air envelopes.

14. The apparatus for identifying leak of face mask of claim 1, wherein the face mask is a loose-fitting face mask, the loose-fitting face mask comprising a surgical mask, a dust mask, or a cloth mask.

15. The apparatus for identifying leak of face mask of claim 1, wherein the face mask is a tight-fitting face mask, the tight-fitting face mask comprising a N95, N99, N100, P95, P99, P100, R95, R99, R100 respirator, an Air-purifying Respirator, a Powered Air-Purifying Respirator (PAPR), a Supplied-Air Respirator (SR) or a Self-Contained Breathing Apparatus (SCBA).

16. The apparatus for identifying leak of face mask of claim 3, wherein the inner space includes:
a first portion, filled with at least one filling material, one side of the first portion being adapted to be connected to the face mask; and
a second portion, being adjacent to another side of the first portion, an interior of the first portion provided with a reservoir, constructed to store the at least one filling materials, wherein the inner space is designed such that material is not interchangeable between the first portion and the second portion, wherein the at least one filling material in the reservoir can moves outward using capillary phenomenon.

17. An apparatus for identifying leak of face mask comprising:
an inner layer, adapted to be close to a wearer's face; and
an outer layer, located on an opposite side of the inner layer, an inner space being between the inner layer and the outer layer, the inner space comprising:
at least one first portion, filled with the at least one filling material, one side of the first portion being adapted to be connected to a face mask; and
at least one second portion, being adjacent to another side of the first portion;
wherein the at least one first portion and the at least second portion are partitioned by partition materials, and the partition material is a permeable material.

18. The apparatus for identifying leak of face mask of claim 17, wherein area of each second portion is equal to or smaller than area of each first portion.

19. A method for identifying leak of face mask, executed by the apparatus for identifying leak of face mask of claim 4, the method comprising:
Step S11: connecting the apparatus for identifying leak of face mask to the face mask;
Step S12: putting the face mask on a wearer's face until attaching to the wearer's face;
Step T11: determining if the second portion is thoroughly filled with the filling materials from the first portion; if the determination result is yes, it is a no-leak condition; if the determination result is no, it is a leak condition, and then proceed to step T12;
Step T12: identifying an unfilled position as the leak location.

20. The method for identifying leak of face mask of claim 18, wherein the determination in Step T11 and/or Step T122 is performed by eyes or by scanning the wearer's face through an image detector.
